(19) United States
(12) Patent Application Publication
(10) Pub. No.: US 2018/0140206 A1
(43) Pub. Date: May 24, 2018

(54) MEDICAL IMPLANT AND SYSTEM FOR INTRAVASCULAR IMPLANTATION

(71) Applicant: BIOTRONIK SE & CO. KG, BERLIN (DE)

(72) Inventor: ANDREAS BITZER, ZUERICH (CH)

(21) Appl. No.: 15/816,065

(22) Filed: Nov. 17, 2017

(30) Foreign Application Priority Data
Nov. 23, 2016 (EP) 16200256.2

(51) Int. Cl. A61B 5/0215; A61N 1/375; A61M 25/09

(52) U.S. Cl. CPC A61B 5/0215; A61M 25/09; A61N 1/37512

(57) ABSTRACT

A medical implant for intravascular implantation has an implant housing and at least a first and a second conveying element. The first and the second conveying elements are arranged on the implant housing in one plane. The first conveying element is mounted rotatably about a first rotation axis and the second conveying element is mounted rotatably about a second rotation axis.

### CROSS-REFERENCE TO RELATED APPLICATION

This application claims the benefit, under 35 U.S.C. § 119, of European patent application EP 16200256.2, filed Nov. 23, 2016; the prior application is herewith incorporated by reference in its entirety.

### BACKGROUND OF THE INVENTION

### Field of the Invention

The invention relates to a medical implant, in particular miniaturized implants implanted in the cardiovascular system, and systems for intravascular implantation thereof.

An example of implants of this kind is provided by implantable pressure sensor devices, in particular a miniature pressure sensor device, which can be implanted in an animal or human body. Pressure sensor devices including microelectromechanical systems (MEMS) and associated chip systems are known in the art. For example, pressure sensor devices with MEMS chips are used for electrical implants for improving the heart function of a patient. Chip systems which can be used for these purposes on the one hand have to deliver sufficiently precise measurement results, and on the other hand must have very small dimensions, so that they can be implanted without difficulty and do not limit the physiological activity of the organism, for example for blood pressure measurements in the pulmonary artery.

Another example of such an implant is provided by pacemaker implants which are conveyed intravascularly for implantation so as to ultimately be implanted in a blood vessel, for example miniaturized cardiac pacemakers without long electrode leads (so-called ‘leadless’ pacemakers).

A technical challenge in conjunction with such implants is that of conveying the implant through the cardiovascular system. In spite of miniaturization of the implant dimensions, difficulties can occur when conveying the implant through the cardiovascular system, in particular if the implant has to be passed through narrow, winding blood vessels having a small bend radius. A second problem is the placement and reliable fixing of the implant at the implantation site, i.e. in contact with the blood flow. A stationary fixing of the implant is necessary for this purpose, without significant disruption to the blood flow. An uncomplicated implantation system and method as well as a simple possibility for explanation are desirable.

U.S. patent publication No. 2016/0082270 A1 describes a fixing unit which is configured to anchor a medical implant in a patient.

The prior art presents systems based on wire loops that are attached to the sides of the implant and that hold the implant in a blood vessel. Systems of this kind, however, have a number of disadvantages: The wire loops are relatively tightly curved in the implanted state, thus heavily stressing the material. Furthermore, the wire loops take up a relatively large amount of space and the connection of the loops to the housing, which at the same time constitutes an electrical bushing, can only be produced in a complex manner.

Stent-based solutions for the securing of implants in a blood vessel are also known. Stent-like solutions have the great advantage that they are relatively rigid and therefore are difficult to guide through blood vessels. In addition, stent systems tend to migrate, i.e. there is the possibility that the implant, over time, will not remain at the implantation site, and instead ‘migrates’ in the blood vessel. Stent systems and associated implantation systems are also complex and costly to produce.

### SUMMARY OF THE INVENTION

One object of the present invention is therefore to provide a medical implant which can be conveyed in a simple manner through curved blood vessels of the cardiovascular system. A further object of the present invention lies in providing a medical implant which can be implanted in the cardiovascular system in a permanent and stationary manner. A further object can also be considered that of providing a medical implant which can be implanted and explanted in a simple way.

Another object of the present invention is to provide systems for implanting a medical implant of the aforesaid type, which systems have an uncomplicated structure and in addition are easy to use and easy to produce.

A further object can be considered that of providing a medical implant which, after implantation in the cardiovascular system, does not significantly adversely affect or hinder the blood flow.

In accordance with the present invention, a medical implant for intravascular implantation is proposed, which implant has an implant housing and at least a first and a second conveying element, which are arranged in one plane on the implant housing. Here, the first conveying element is mounted rotatably about a first rotation axis, and the second conveying element is mounted rotatably about a second rotation axis.

In conjunction with the present invention, any types of medical implants which are implanted intramuscularly and/or have to be conveyed through blood vessels during the implantation process are conceivable. Examples of these are cardial implants, for example pacemakers, in particular pacemaker implants without electrode leads (what are known as ‘leadless’ implants) for implantation in a chamber of the heart, cranial implants, stent or coil implants, and the like. One embodiment provides a miniaturized pressure sensor for implantation in the pulmonary artery as medical implant, so as to be able to take blood pressure measurements over a longer period of time.

The implant housing may be made of material which is biocompatible and at the same time robust with respect to influences in the surrounding environment of the implantation site. A suitable material is titanium, for example.

The housing geometry may be selected in such a way that it is possible for the implant to be conveyed through a blood vessel or implanted in a blood vessel in a simple manner.

In the context of the invention, a conveying element is understood to mean a component which can be used to simplify the conveying of the implant through blood vessels to the implantation site. In accordance with the invention, the conveying elements may be rotatably mounted. The first conveying element and the second conveying element may be arranged in the same plane on the implant housing. The first conveying element and the second conveying element may be used in order to convey or transport the medical implant inside a body of a subject, e.g. intramuscularly or intravascularly. Thus, the first conveying element and the second conveying element may also be called the first transporting element and the second transporting element, respectively.

In accordance with an embodiment of the present invention, the implant housing has a shape with a proximal end and a distal end, wherein the first conveying element is arranged close to the proximal end and the second conveying element is arranged close to the distal end. Here, the first and the second conveying element may be arranged on at least one side outer surface of the implant housing.

A suitable geometric basic shape of the outer housing is a cube, for example; a cylinder, an ellipsoid, or any bar-like elongate shape is also conceivable. Here, shapes with rounded corners and edges would be advantageous, so as to reduce the flow resistance, so that the implantation process is simplified and thrombus formation at the implant is hindered. The shape can have defined flat outer surfaces, as is the case with a cube, and curved outer surfaces, as is the case with an ellipsoid or the lateral surface of a cylinder. If the medical implant for example has a cuboid outer shape, the conveying elements can be arranged on a side outer wall, wherein the first conveying element is arranged close to the proximal end of the implant housing, and the second conveying element is arranged close to the distal end of the implant housing. Here, the conveying elements can be formed by parts of the implant housing or can be connected to the implant housing by a screwed, welded, soldered or glued connection, or the like. If the implant housing a cylindrical with a lateral surface and two base faces, the at least two conveying elements can be arranged along a line on the lateral outer surface running parallel to the height of the cylinder.

Within the scope of this application, proximal and distal are understood in relation to the operator. The proximal end of the implant is closest to the operator during implantation, and the distal end is distanced furthest from the operator during the insertion of the implant.

The two points at which the first and second conveying elements are arranged may have a relatively large distance from one another and may be connected to one another directly where possible, wherein this is to be understood in the sense that a path leading along the outer housing should run from the first point to the second point as directly as possible and in the straightest line possible. In this way, it is achieved that a guide apparatus for implantation, for example a guide wire or the like, can be guided easily and directly from the first to the second conveying element and can be coupled to the conveying elements, wherein the geometry of the implant housing is adapted to the guide wire or nestles against the guide wire to the greatest possible extent.

In one embodiment of the present invention the first and second rotation axes may be oriented parallel to one another. Furthermore, the first and second rotation axes can be configured in such a way that they are oriented perpendicular to the longitudinal axis of the implant housing. Alternatively or additionally, the rotation axes can be oriented perpendicular to the plane in which the first and second conveying element are arranged.

If the first and the second conveying element are coupled to a guide apparatus for implantation, for example a guide wire or the like, the rotatably mounted conveying elements thus provide a certain degree of movement of the implant relative to the guide apparatus. This is advantageous in the case of implantation, since the implant is not rigidly coupled to the guide apparatus and can therefore be conveyed more easily through blood vessels having a small diameter and small bend radius.

With the orientation of the rotation axes, it can be achieved that the length of the implant is oriented substantially tangentially to the curve of the bend of the portion of the guide apparatus at which the medical implant is disposed.

Within the scope of this application, the longitudinal axis is understood to mean the axis along which the implant has its greatest extent.

In accordance with one embodiment according to the invention, the first and the second conveying element may be configured to interact with a guide wire.

In the sense of the invention, in this regard, other apparatuses which can be used in order to intravascularly convey implants, such as catheter systems or the like, are also included besides a guide wire. In accordance with the invention, the interaction of the conveying elements with the guide wire is understood to mean any form of a coupling making it possible to convey the medical implant by means of the guide wire.

In one embodiment of the invention, the conveying elements may each have a holder member, wherein the holder member is configured to receive a guide wire. Here, the holder member may have the form of a ring, an eyelet, a loop, or a hook.

The guide wire can thus be threaded for example through ring-like or eyelet-like holder members. In the case of loops and hooks, the guide wire is clamped or hooked respectively, wherein a mechanism which secures the coupling, for example by latching or engagement, can be used. Here, the holder members should be movable along the guide wire, in other words the holder members should not be rigidly connected to the guide wire. Other embodiments for the holder members are also conceivable, which likewise offer a coupling of the described kind between conveying elements and the guide wire, for example rail, bracket or tunnel systems or the like.

In an embodiment of the invention the medical implant may have a fixing structure with at least two holder units, wherein the fixing structure can adopt an expanded state and a collapsed state. Here, the fixing structure may be configured in the expanded state to fix the medical implant at an implantation site.

In one embodiment of the invention the holder unit may have the form of a ring, an eyelet, a loop, or a hook.

For example, the fixing structure can be embodied in the form of legs, flaps, a framework, lattice, mesh, or stent.

In the sense of the invention a collapsed state of the fixing structure is understood to mean a form in which the fixing structure has an outer geometry suitable for intravascular conveyance. An expanded state is understood to mean a form in which the fixing structure has an outer geometry suitable for fixing/anchoring/securing the fixing structure itself and also the medical implant in a blood vessel.

If the fixing structure is embodied as legs, flaps or a framework, these are in the collapsed state when they are closed and are in the expanded state when they are folded out. The same is true for a lattice-like, mesh-like or stent-like fixing structure, which can assume the collapsed and expanded state by folded up or unfolded respectively.

The fixing structure may be made of biocompatible material suitable for permanent implantation in the blood flow. After reaching the implantation site, the fixing structure can be expanded. The holder units are coupled to the fixing structure and are configured to hold the fixing structure in the collapsed state.

The holder units can be fixedly connected to the fixing structure. For example, they are arranged on a proximal end and/or distal end of the fixing structure and may have the form of a ring or are similar to a ring. Rail, bracket or tunnel systems or the like are also conceivable.

In one embodiment the holder units can be connected to a cord or the like. This would simplify an explantation of the implant, for example in that the cord is caught over a catheter.

In accordance with one embodiment of the invention, the holder units of the fixing structure may be configured to interact with a guide wire so that the fixing structure is in the collapsed state.

The holder units may be configured so that they can be coupled to the guide wire in such a way that the fixing structure is held in the collapsed state. To this end, the guide wire can be received for example by the holder units.

The guide wire can thus also be threaded for example through ring-like or eyelet-like holder units, similarly to the holder members. In the case of loop-like or hook-like holder units, the guide wire is clamped or hooked respectively, wherein a mechanism which secures the coupling, for example by latching or engagement, can be used. Here, the holder units should be movable along the guide wire, i.e. the holder units should not be rigidly connected to the guide wire. Other embodiments for the holder units are also conceivable, which likewise offer a coupling of the described type between conveying elements and the guide wire, for example rail, bracket or tunnel systems or the like.

In one embodiment of the invention the fixing structure may comprise at least two limbs made of flexible material. Here, the limbs are closed in the collapsed state and are folded out from one another in the expanded state. Furthermore, it is conceivable that each limb has a meandering and/or zigzagging portion.

The limbs may be configured, in the collapsed state, to provide an extension of the implant housing so that the overall diameter of the medical implant is not increased or is only slightly increased. At a desired implantation site within a blood vessel, the limbs can be brought into the expanded state. The overall diameter of the implant is then significantly increased, and the limbs are folded out from one another and each contact the inner wall of the vessel at one of their ends, so that the force can be transferred to the inner wall of the vessel (for example by wedging or pressure) in order to support the medical implant in the blood vessel and hold it at that point. For example, the unfolded limbs form a V shape and fix the implant in the blood vessel in that shape. A V shape is characterized in that it has an anisotropic flow resistance, i.e. an implant fixed in the blood vessel puts up a resistance to the blood flow, which resistance is dependent on the direction of blood flow. By corresponding placement of the implant, the implant can thus be prevented from ‘migrating’ after a certain time, i.e. it can be prevented from moving in a direction with the blood flow. In this regard, other forms for the fixing structure suitable for the aforementioned purpose with anisotropic flow resistance in the expanded state are also conceivable.

In accordance with one embodiment of the invention the fixing structure may be made at least in part a flexible material, since the implant can be conveyed through blood vessels with lower resistance during the implantation, and, after fixing of the implant in the blood vessel, a certain adaptation of the limbs to the cyclic forces occurring due to the blood flow in the vessel takes place, so that the service life of the material is extended. The degree of flexibility should be selected in accordance with the described properties to be satisfied.

In accordance with one embodiment, the proximal ends of the limbs may have a meandering portion, which may be the portion that contacts the inner wall of the vessel. The contact face of the fixing structure on the inner wall of the vessel is thus increased, and the pressure applied by the fixing structure to the inner wall of the vessel is thus reduced. Furthermore, the elasticity of the fixing structure in the region of the contact faces is increased. It is thus achieved that the contact faces run tangentially to the vessel wall. This is advantageous since on the one hand the holding of the implant in the vessel is increased and on the other hand the risk of the limbs cutting into the vessel wall decreases. Other shapes and patterns for the portion of the limbs that provide the effect are also included, for example zigzag shapes, step shapes or other repetitive geometries.

In accordance with an embodiment of the present invention, the fixing structure may be coupled to at least one mechanical spring. Here, the mechanical spring may be tensioned when the fixing structure is in the collapsed state and may be in the rest position when the fixing structure is in the expanded state.

The mechanical spring according to the invention can be provided for example in the form of a leg spring, tab spring, helical spring or similar spring. For example, the spring can be arranged on or integrated in or inside the implant housing. If the fixing structure has legs or limbs as in the described embodiments, the legs or limbs can be coupled at their end facing the implant housing to the spring or can transition into a spring. In the described embodiment with two limbs which fold out in a V shape, the spring can be arranged between the two limbs (i.e. at the apex of the V), wherein the spring is secured to an end of the implant housing. In one embodiment, the limbs each have a holder unit at the proximal end, by which they can receive a guide wire. For this purpose, the limbs have to be closed, so that the holder units are oriented in a line and the guide wire can be received. In order to close the limbs, the mechanical spring is tensioned, and the fixing structure is in the collapsed state. If the guide wire is retracted, so that it is no longer received by the holder units, the fixing structure immediately adopts the expanded state, for example by folding out the two limbs into the V shape, caused by the restoring force of the spring, which moves the fixing structure back into the rest position.

In accordance with one embodiment of the invention, the fixing structure may be made at least in part or completely of a shape-memory material, in particular a shape-memory alloy such as nitinol.

Shape-memory materials are characterized in that they can be deformed under specific external circumstances, but after deformation can resume their initial form by means of a stimulus. For example, a stimulus of this kind can be constituted by a change in temperature.

For example, the shape-memory material can be selected in such a way that it resumes its initial form at body temperature (‘austenite state’). The expanded state may be selected as the initial form for the fixing structure. The fixing structure, prior to implantation, is first converted into the deformable state (‘martensite state’, for example by cooling), shaped into the collapsed state, and coupled to a guide wire, so that the holder units receive the guide wire. In the event of implantation, the shape-memory material is heated again by the body temperature to a temperature above the transition temperature. Here, the fixing structure is still held in its collapsed state by the guide wire, this state being referred to as the stress-induced martensite state. As soon as the implant has been conveyed to the desired implantation site and the guide wire has been removed, the guide structure regains its initial form (the expanded state, or austenite state).

The use of a shape-memory material for the fixing mechanism enables its change from the collapsed to the expanded state without use of an additional mechanical energy store.

In an embodiment of the present invention the medical implant may have a gripper unit. The gripper unit can be embodied for example as a ring, eyelet, hook or the like and can be arranged at the proximal end of the medical implant. Here, the gripper unit serves to attach the implant to a component for pushing or pulling the implant along the guide wire. The gripper unit can also be used after implantation for adjustment of the implantation site, or for explantation, by being grasped or held by a catheter or the like, so that the medical implant can be moved.

The present invention also contains a system for intravascular implantation of a previously described medical implant, wherein the system has at least one flexible guide wire and an elongate slider unit. Here, the implant, the guide wire and the slider unit may be arranged in such a way that

a) the guide wire interacts with the first and second conveying element of the implant, wherein the first and second conveying element each may have a holder member, which holder members received the guide wire; and
b) the slider unit interacts with the guide wire, so that the slider unit can be moved along the guide wire, wherein the slider unit is designed to push and/or to pull the medical implant along the guide wire.

In another embodiment of the system according to the invention for intravascular implantation, the slider unit may contain a flexible shaft, referred to hereinafter as a slider shaft, and a slider module, wherein the slider shaft surrounds a lumen and the slider module is arranged at the proximal end of the slider shaft. Here, the guide wire may be arranged at least in part within the lumen and the slider module interacts with the medical implant in such a way that the implant can be at least held, pulled and/or pushed by the slider module. In this way, by means of the slider unit, the position of the implant in relation to the guide wire can be controlled by the slider module.

The holder members may receive the guide wire so that the implant is coupled to the guide wire, but can move freely there along. For example, a coupling in which the holder members are configured as ring-, eyelet-, loop-, hook-, rail-, bracket- or tunnel-like members or are designed in a form similar to those described can also be implemented.

A system for intravascular implantation which makes it possible to convey implants through blood vessels having a small diameter and small bend radius in a simple manner is created by the system according to the invention and described embodiments. In addition, the system has an uncomplicated structure and can be preassembled by the manufacturer, thus sparing a laborious and costly assembly directly on site at the time of implantation.

In one embodiment of the system according to the invention, the medical implant and the guide wire may be arranged in such a way that the fixing structure is arranged in the proximal position and the implant housing is arranged in the distal position. Alternatively, the implant housing can be arranged in the proximal position and the fixing structure can be arranged in the distal position.

The slider unit can be embodied as a pusher catheter, which for example is used typically for the catheterization of the cardiovascular system. A system which can grip and hold the medical implant via the gripper unit in order to push or pull the implant can also be selected as slider unit. In this way, the operator is provided at the time of implantation with greater freedom for conveying and placing the implant, and in particular the adjustment of the implant position is thus simplified. If the implant is arranged on the guide wire so that the fixing structure is in the proximal position and the implant housing is in the distal position, the fixing structure itself, parts thereof or the holder units can thus be used as gripper units. If, by contrast, the implant housing is arranged in the proximal position and the fixing structure is arranged in the distal position, the gripper unit can be arranged for example at the proximal end of the implant housing, which can be gripped and held by the slider unit. A slider unit of this type with gripping function can be configured in cooperation with the guide wire in such a way that the grip is released when the guide wire is retracted.

In one embodiment of the described system for intravascular implantation of a described medical implant, the system does not comprise a slider unit. Instead, the system has a long flexible shaft which surrounds a lumen and has a proximal end and a distal end. The implant and the guide wire are arranged at least in part within the lumen, wherein the guide wire is received by the conveying elements. Here, the implant may be arranged close to the distal end of the flexible shaft, wherein the fixing structure of the implant is oriented towards the distal end of the flexible shaft, that is to say in the direction in which the shaft is advanced in the vascular system. The fixing structure is held here in the collapsed state by the guide wire. The flexible shaft, together with guide wire and implant in the inner lumen, is advanced intravascularly to the implantation site, where the implant is released from the flexible shaft with the aid of the following process. The implant within the flexible shaft is advanced using the guide wire in the distal direction until the fixing structure protrudes in part from the flexible shaft. The implant housing should still be disposed within the flexible shaft. The guide wire is then retracted in the proximal direction, so that the fixing structure is brought into the expanded state, so that the distal part of the fixing structure is supported against the inner wall of the vessel, wherein the proximal part of the fixing structure is arranged within the flexible shaft. The flexible shaft is then retracted in the proximal direction, wherein the implant is released from the flexible shaft and is held at the implantation site by the fixing structure. In this way, a sudden release of the implant is prevented and a controlled release from the flexible shaft is made possible.

In accordance with an embodiment according to the invention of the system for intravascular implantation, the medical implant may have a previously described fixing structure with at least two holder units as described before, wherein the fixing structure can adopt an expanded and a collapsed state. Furthermore, the holder units are configured to interact with the guide wire in that the guide wire is received by the holder units, so that the fixing structure is in the collapsed state. Here, the fixing structure is configured to transition into the expanded state as soon as the guide wire ceases to interact with the holder units.

In accordance with one embodiment, the holder units may be configured in the form of rings, eyelets, loops, hooks, rails, brackets, tunnels or in a form similar to those described. Similarly to the holder members, the holder units may also be configured so that they ensure a coupling of the fixing structure to the guide wire, wherein it should be possible however that the fixing structure and with it the medical implant can be moved along the guide wire. The fixing structure may be held in the collapsed state when the holder units are coupled to the guide wire. As soon as this coupling is cancelled, for example in that the guide wire is retracted, the fixing structure adopts the expanded state.

Other features which are considered as characteristic for the invention are set forth in the appended claims.

Although the invention is illustrated and described herein as embodied in a medical implant and a system for intravascular implantation, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1A is a diagrammatic, side view of a first embodiment of a medical implant according to the invention;

FIG. 1B is a perspective view of the first embodiment of the medial implant;

FIGS. 2A and 2B are side views of an exemplary embodiment of a fixing structure;

FIGS. 3A and 3B are side views of further exemplary embodiments of the fixing structure;

FIGS. 4A-4E are perspective views of exemplary embodiments of conveying elements and holder members;

FIGS. 5A-5C are illustrations showing an exemplary process for changing between a collapsed and expanded state for an embodiment of the medical implant;

FIGS. 6A-6D are illustrations of a further exemplary implantation process for an embodiment of the medical implant;

FIGS. 7A-7C are illustrations showing embodiments of a system for intravascular implantation of the medical implant;

FIG. 8 is a perspective view of an embodiment of a slider unit of the system for intravascular implantation of a medical implant;

FIG. 9 is an illustration of an embodiment of the system for intravascular implantation of the medical implant within a bend having a small bend radius;

FIG. 10 is a perspective view of a detail of an embodiment of the fixing structure for the system for intravascular implantation of a medical implant; and

FIGS. 11A-11C are illustrations of another embodiment of the medical implant and the system for intravascular implantation of the medical implant.

### DETAILED DESCRIPTION OF THE INVENTION

In the drawings, functionally like or identically acting elements are referenced in each case by the same reference signs. The drawings are schematic depictions of the invention. They do not show specific parameters of the invention. The drawings also present merely typical embodiments of the invention and are not intended to limit the invention to the depicted embodiments.

The embodiments and/or features of the medical implant according to the invention and of the system for intravascular implantation of an implant of this kind depicted in the drawings can also be combined with one another and are not intended to be limited to the example and features thereof shown in the drawings.

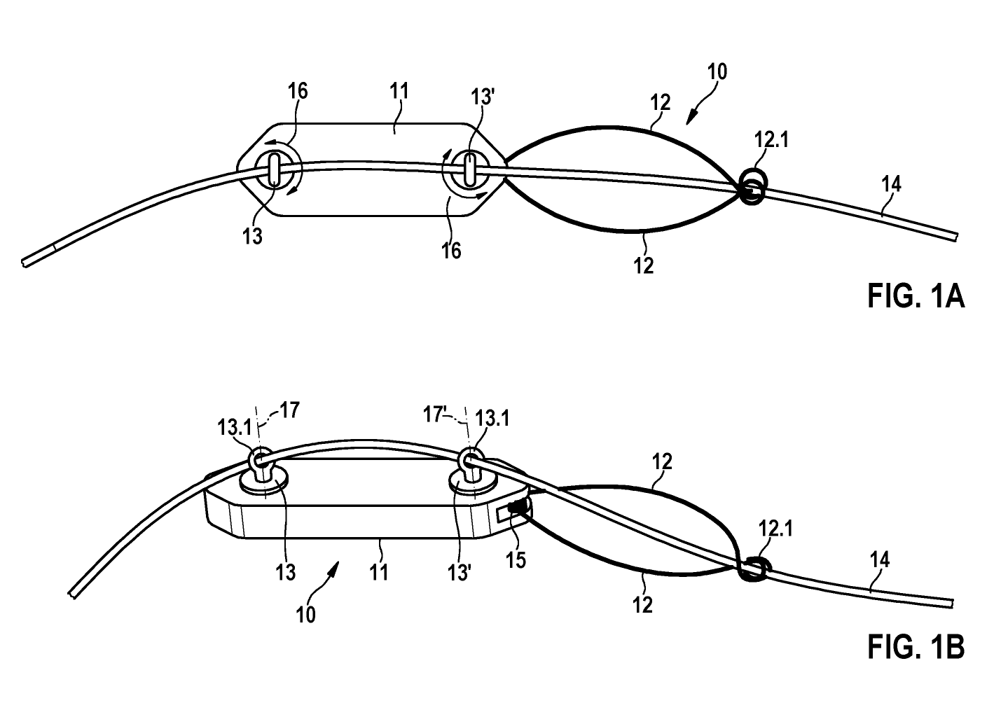

Referring now to the figures of the drawings in detail and first, particularly to FIGS. 1A and 1B thereof, there is shown a first embodiment of a medical implant in a side view (FIG. 1A) and an oblique view (FIG. 1B). Here, a medical implant 10 has an implant housing 11 and an embodiment of a fixing structure 12 with two limbs, with holder units 12.1 arranged at the distal ends of the limbs. The proximal ends of the two limbs of the fixing structure 12 are coupled to a mechanical spring 15, which is arranged on the implant housing 11. Two conveying elements 13, 13′ with two holder members in the form of eyelets 13.1 are arranged on a side face of the implant housing. The two conveying elements 13, 13′ are mounted here rotatably in the rotation direction 16 about rotation axes 17, 17′. A guide wire 14 is received by the holder members 13.1 and the holder units 12.1, so that the fixing structure is held in the collapsed state and so that the medical implant 10 can move along the guide wire 14.

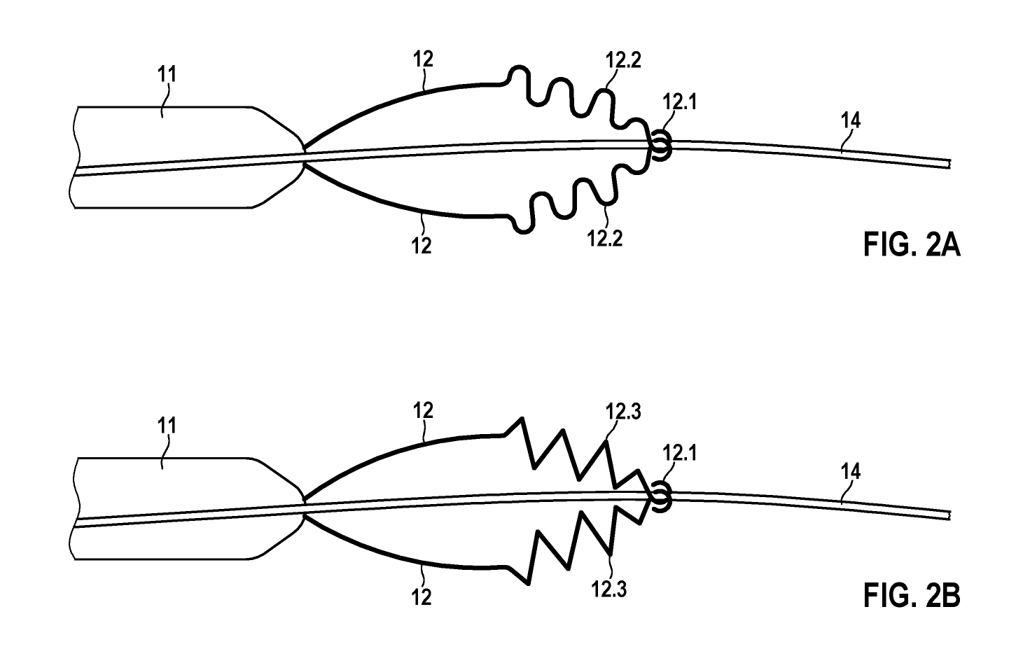

FIGS. 2A-2B show two exemplary embodiments of the fixing structure. The end of the implant housing 11 coupled to the fixing structure 12 and an exemplary embodiment of the fixing structure 12 with two limbs are illustrated, wherein the guide wire 14 is received by the holder units 12.1 at the ends of the fixing structure 12 leading away from the implant housing 11. Here, in FIG. 2A, the two limbs of the fixing structure 12 each have a meandering portion 12.2; in FIG. 2B the two limbs of the fixing structure 12 each have a zigzagging portion 12.3.

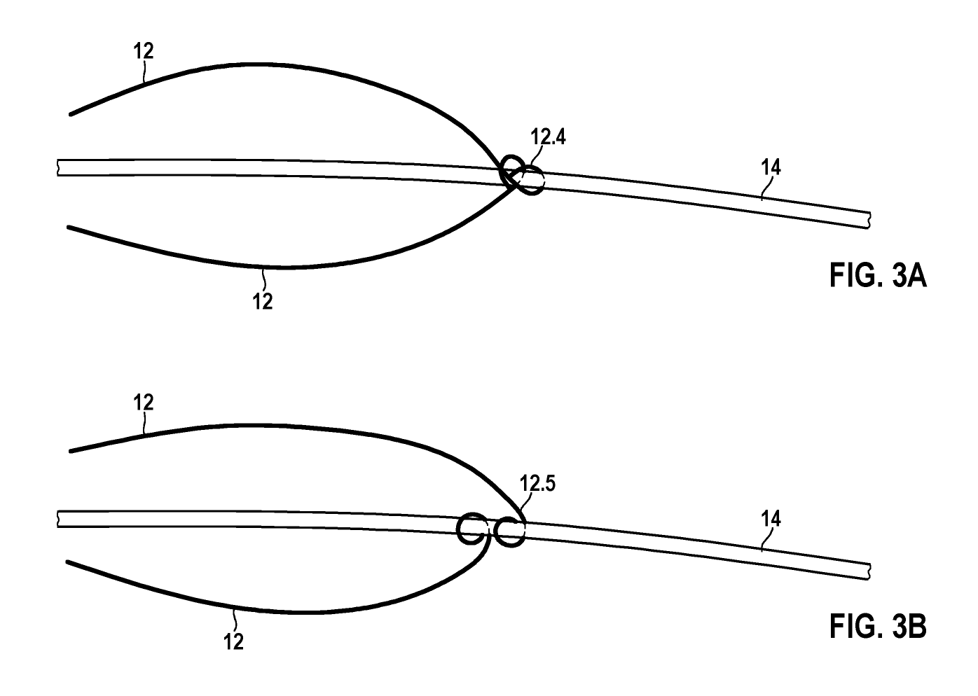

FIGS. 3A and 3B shows further exemplary embodiments of the fixing structure. In FIGS. 3A and 3B the guide wire 14 is received by holder units 12.4 and 12.5 of the fixing structure 12, so that the fixing structure 12 is in the collapsed state. Here, in FIG. 3A, the holder units are illustrated in the form of eyelets 12.4; and in FIG. 3B are illustrated in the form of hooks 12.5.

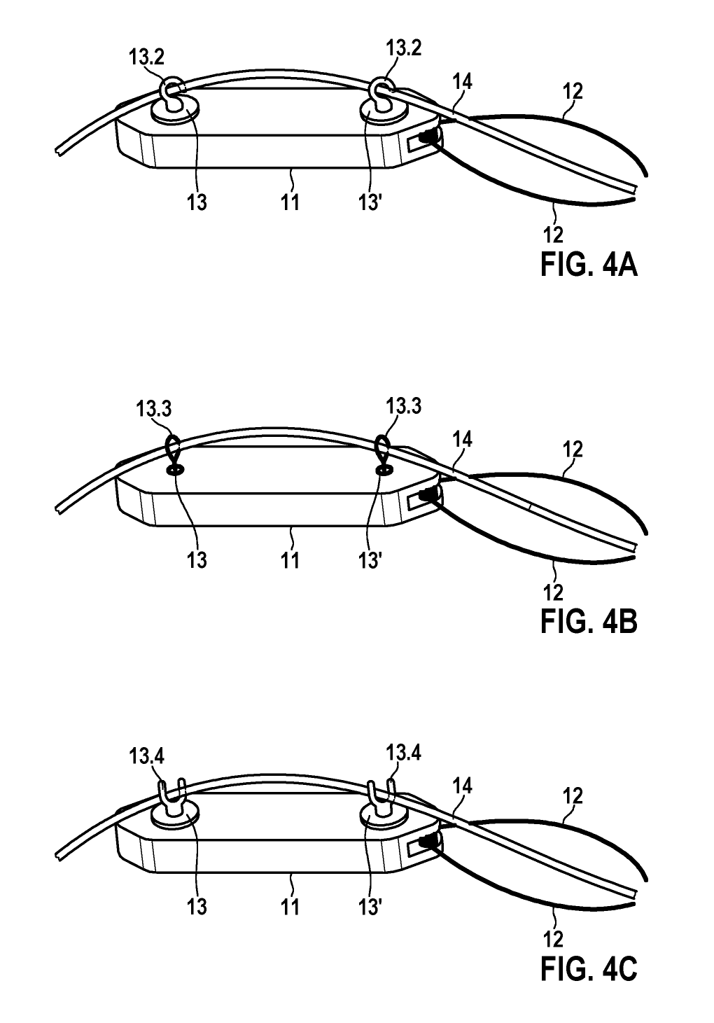

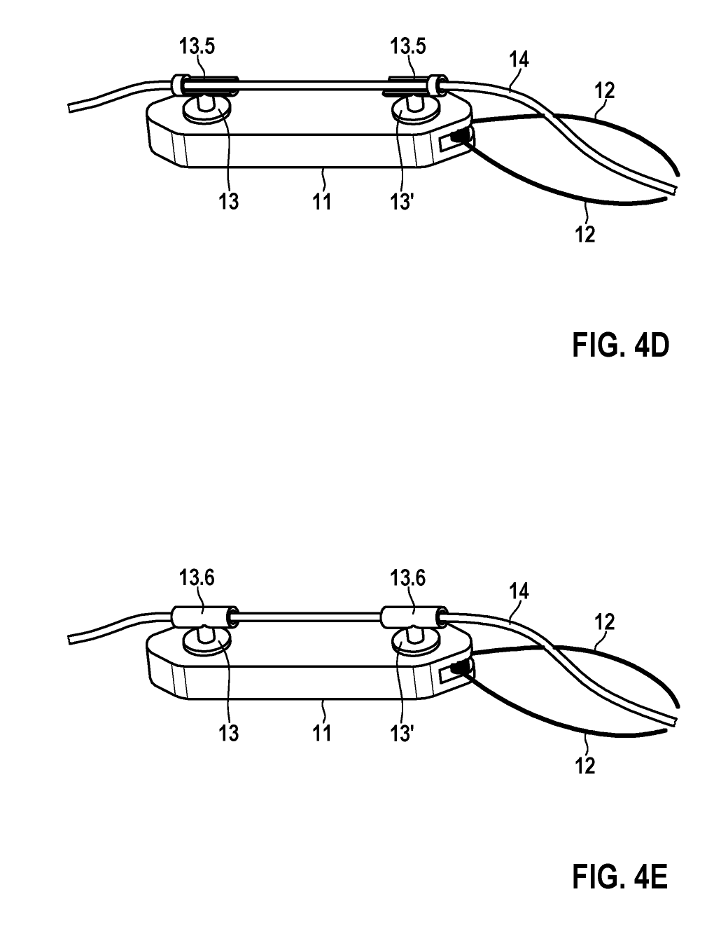

FIGS. 4A-4E show exemplary embodiments of the conveying elements 13, 13′ and holder members 13.2 . . . 13.6. The implant housing 11 is illustrated schematically with two conveying elements 13, 13′ and different embodiments of the holder members 13.2 . . . 13.6, an embodiment of the fixing structure 12 with two limbs in the collapsed state, and the guide wire 14. The guide wire is received by the holder members 13.2 . . . 13.6, so that the medical implant is coupled to the guide wire, but is movable there-along. In the exemplary embodiments the holder members are hook-like holder members 13.2, loop-like holder members 13.3, bracket-like holder members 13.4, rail-like holder members 13.5 and tunnel-like holder members 13.6.

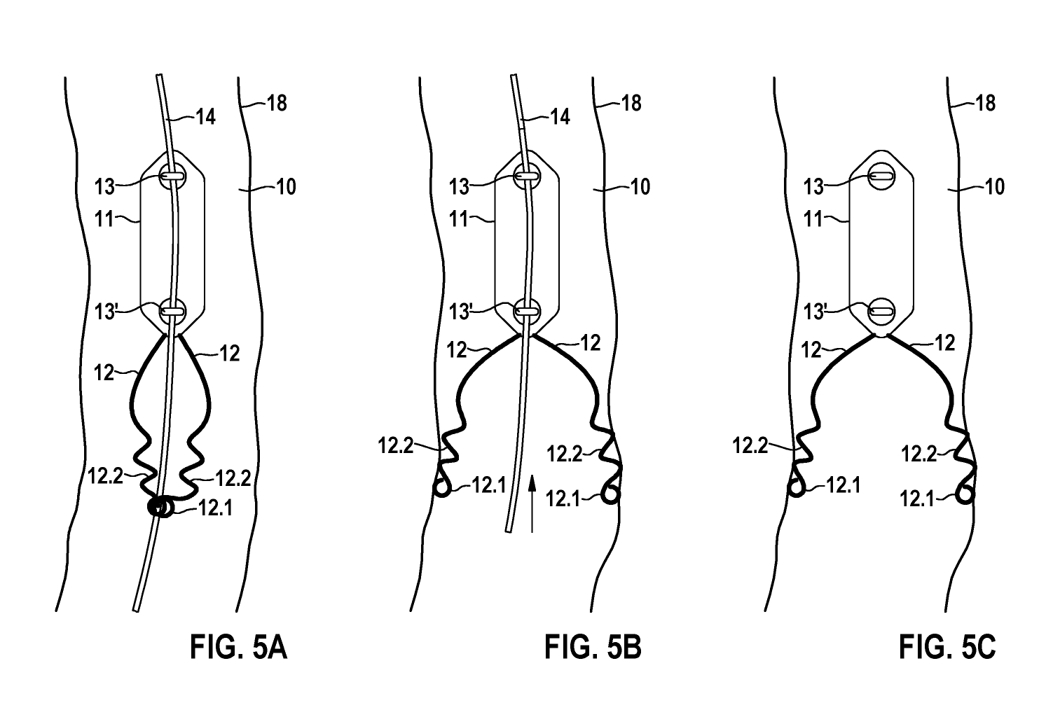

FIGS. 5A-5C show an exemplary process for changing between the collapsed and expanded state for the fixing structure on the basis of an embodiment of the medical implant. Here, FIGS. 5A-5C show the medical implant 10 with implant housing 11 and a possible embodiment of the fixing structure 12 with two limbs, wherein the limbs each have a meandering portion 12.2. Here, the implant is disposed within a lumen 18. In FIG. 5A, the guide wire 14 is received by the holder units 12.1 at the ends of the fixing structure 12 leading away from the implant housing 11 and by the conveying elements 13, 13′ on the implant housing, so that the fixing structure 12 is in the collapsed state and the implant 10 is movable along the guide wire 14. In FIG. 5B the guide wire 14 is removed from the holder units 12.1 (either in the arrow direction or against the arrow direction; in the latter case the guide wire would be removed firstly from the conveying elements 13, 13′ and then from holder units 12.1). As soon as the guide wire 14 is no longer received by the holder units, the fixing structure 12 adopts an expanded state. For this purpose, the two limbs of the fixing structure 12 unfold in the radial direction, wherein the ends of the limbs are supported against the inner wall of the lumen 18 in the shown example at the meandering portions 12.2. The guide wire 14 can then be removed, as illustrated in FIG. 5C, and the implant is fixed in the lumen 18 by the fixing structure 12. In the shown manner, the implant 10 can be implanted for example in a blood vessel.

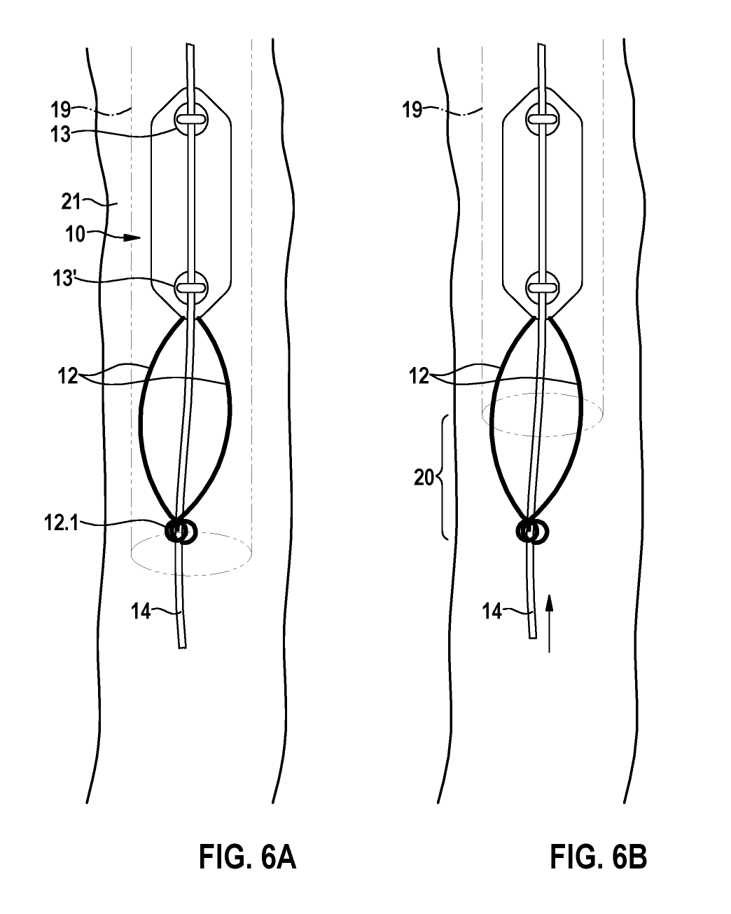

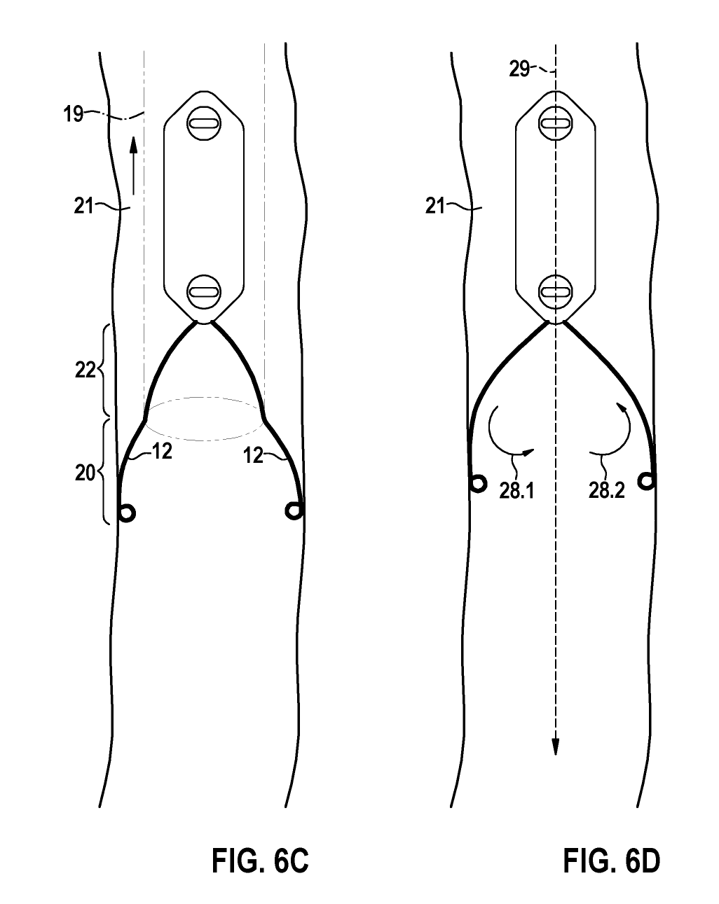

FIGS. 6A-6D show a further exemplary implantation process for an embodiment of the medical implant 10. The implant 10, in FIG. 6A, is disposed in the inner lumen of a flexible shaft 19, wherein conveying elements 13, 13′ and holder units 12.1 receive the guide wire 14, so that the implant 10 is movable along the guide wire 14 and the fixing structure 12 is in the collapsed state. The flexible shaft is advanced into a blood vessel 21 as far as the desired implantation site. In FIG. 6B, the flexible shaft 19 is moved in the arrow direction until at least a portion 20 of the fixing structure 12 protrudes from the flexible shaft 19. In FIG. 6C, the guide wire 14 has been removed, so that the fixing structure 12 adopts the expanded state. The portion 20 of the fixing structure 12 protruding from the flexible shaft 19 is supported against the inner wall of the blood vessel 21. The portion 22 of the fixing structure 12 disposed within the flexible shaft is supported lightly against the inner wall of the flexible shaft 19. The flexible shaft 19 is then removed in the arrow direction, so that the implant 10 is fully released from the flexible shaft and fixed at the implantation site, as illustrated in FIG. 6D. The removal, i.e. retraction, of the shaft 19 should be performed carefully and smoothly, since otherwise the implant 10 could also be entrained. The fixing structure may be designed so that the angles 28.1 and 28.2 enclosed by each of the limbs and the blood flow direction 29 should be smaller than 90°. For example, the limbs can form a V shape. In this way, the implant 10 is provided with an anisotropic flow resistance, i.e. an implant fixed in the blood vessel offers resistance to the blood flow, said resistance being dependent on the blood flow direction. It is thus possible to prevent the implant from ‘migrating’ after a certain period of time, i.e. from being moved in a direction with the blood flow.

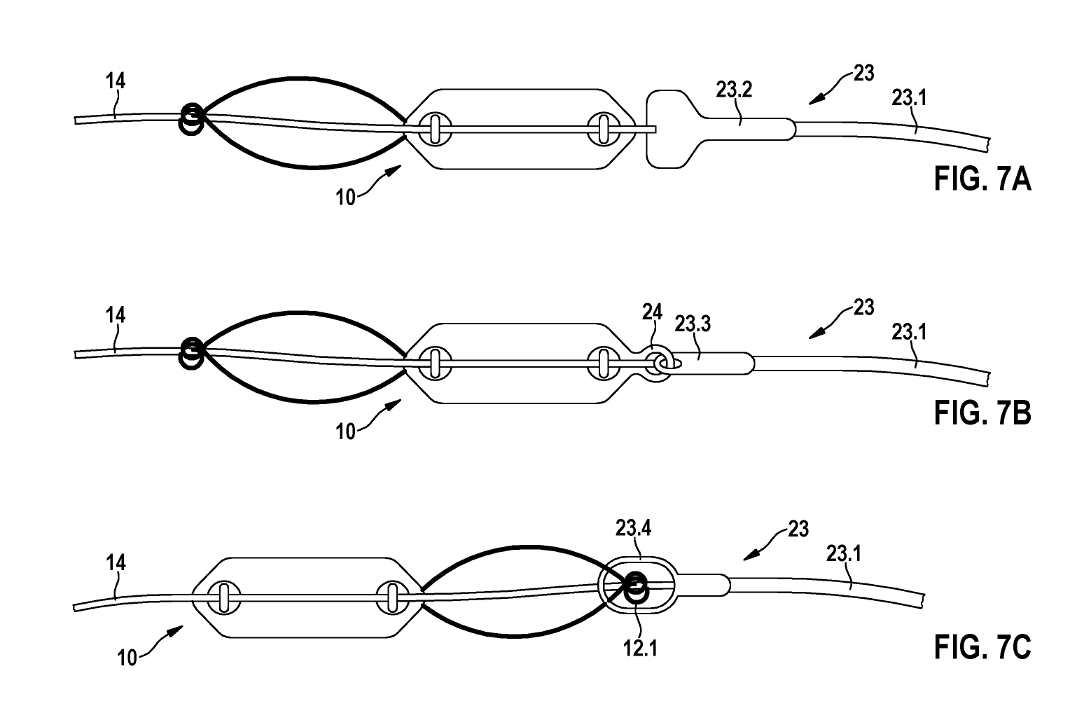

FIGS. 7A-7C show embodiments of the system for intravascular implantation of a medical implant 10, containing a guide wire 14 and a slider unit 23. In the examples the system is shown in arrangements which would be suitable for conveying the implant to the implantation site. FIGS. 7A, 7B and 7C show the implant 10, which interacts with the guide wire so that it can move along the guide wire, and wherein the fixing structure 12 is in the collapsed state. Furthermore, the slider unit 23 is illustrated with a slider shaft 23.1, wherein the latter has an inner lumen in which the guide wire 14 is arranged at least in part. In FIG. 7A the slider unit has a slider module 23.2, which can be used to advance the implant 10. In the exemplary embodiment in FIG. 7B the medical implant has a gripper unit 24, whereas the slider unit 23 is configured so that the gripper unit 24 can be gripped and held via the slider module 23.3. In this way, the implant 10 can then be pushed or pulled via the slider unit 23. In FIG. 7C an exemplary embodiment is shown in which the slider unit 23 has a slider module 23.4 which receives holder units 12.1. It is thus possible to pull or push the implant 10 using the slider unit.

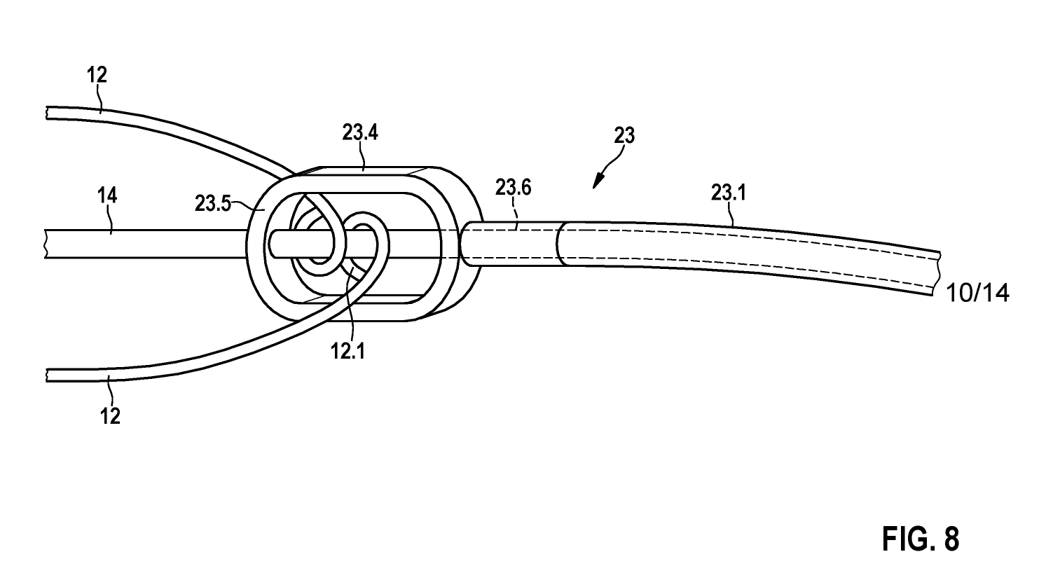

FIG. 8 shows a detailed view of the slider unit from exemplary embodiment of FIG. 7C of the system for intravascular implantation of a medical implant 10. The holder units 12.1 of the two limbs of the fixing structure 12 receive the guide wire 14, so that the fixing structure 14 is in the collapsed state. At the same time, the slider module 23.4 is configured in such a way that it can receive a guide wire 14 in that the guide wire 14 can be introduced into the slider shaft 23.1 via the bushings 23.5 and 23.6. The structure enables the implant 10 to be pulled or pushed by the slider unit 23 by the holder units 12.1.

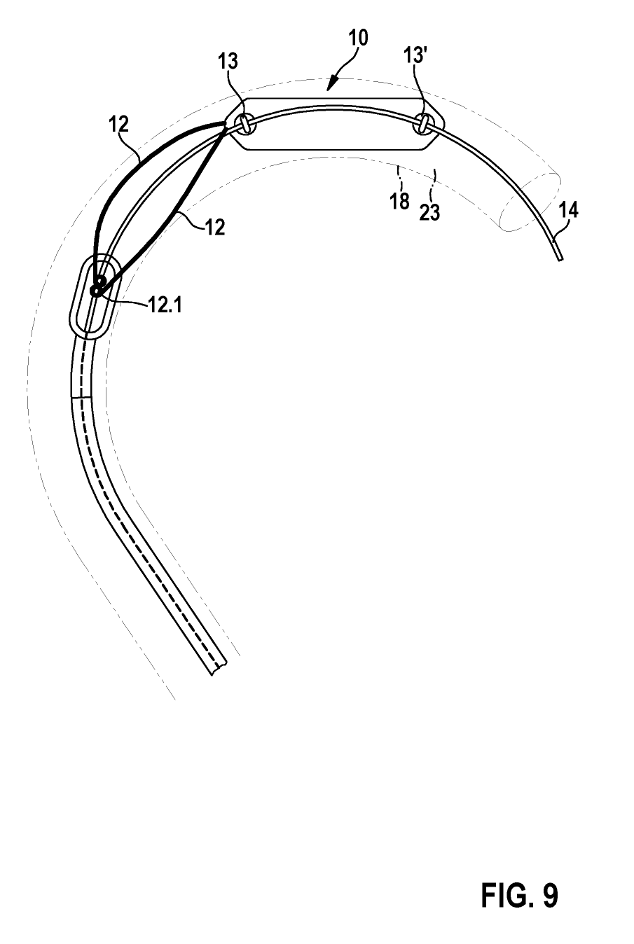

FIG. 9 shows an embodiment of the system for intravascular implantation of a medical implant 10 with slider unit 23 as illustrated schematically in FIG. 7C, within a bend having a small bend radius within a lumen 18. The guide wire 14 passes through the lumen 18, wherein the guide wire 14 is received by the conveying elements 13, 13′ and holder units 12.1 of the fixing structure 12, so that the implant 10 can be pulled or pushed along the guide wire 14 with the aid of the slider unit 23. In FIG. 9 it is schematically illustrated that the length of the implant 10 adapts to the bend of the guide wire 14 on account of the rotatably mounted conveying elements 13, 13′, so that the implant can be conveyed through the curved lumen 18 with less resistance.

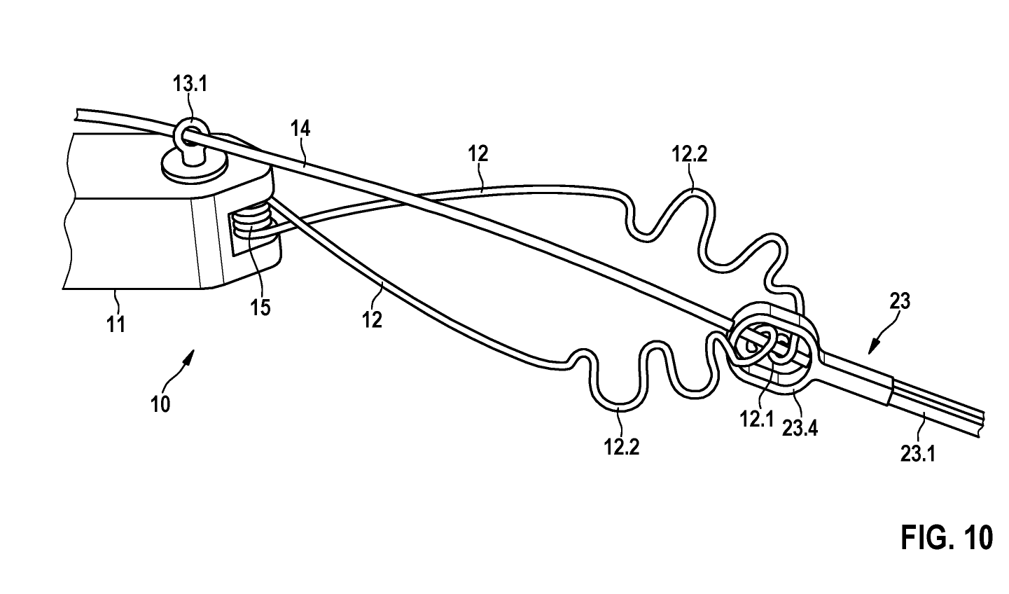

FIG. 10 shows a detailed view of an embodiment of the medical implant 10, in particular the fixing structure 12 and the system for intravascular implantation of a medical implant 10. What are shown are the guide wire 14, the end of the implant housing 11 facing the fixing structure, the fixing structure 12 with two limbs, wherein the two limbs each have a meandering portion 12.2 and are coupled at their end facing the implant housing 11 to a mechanical spring 15, and a slider unit 23 with slider module 23.4 and slider shaft 23.1. Here, the guide wire is received by the holder members 13.1 of the conveying elements 13, 13′ on the implant housing, by the holder units 12.1, and by the slider unit 23, so that the implant can be pulled or pushed along the guide wire 14 by the slider unit 23.

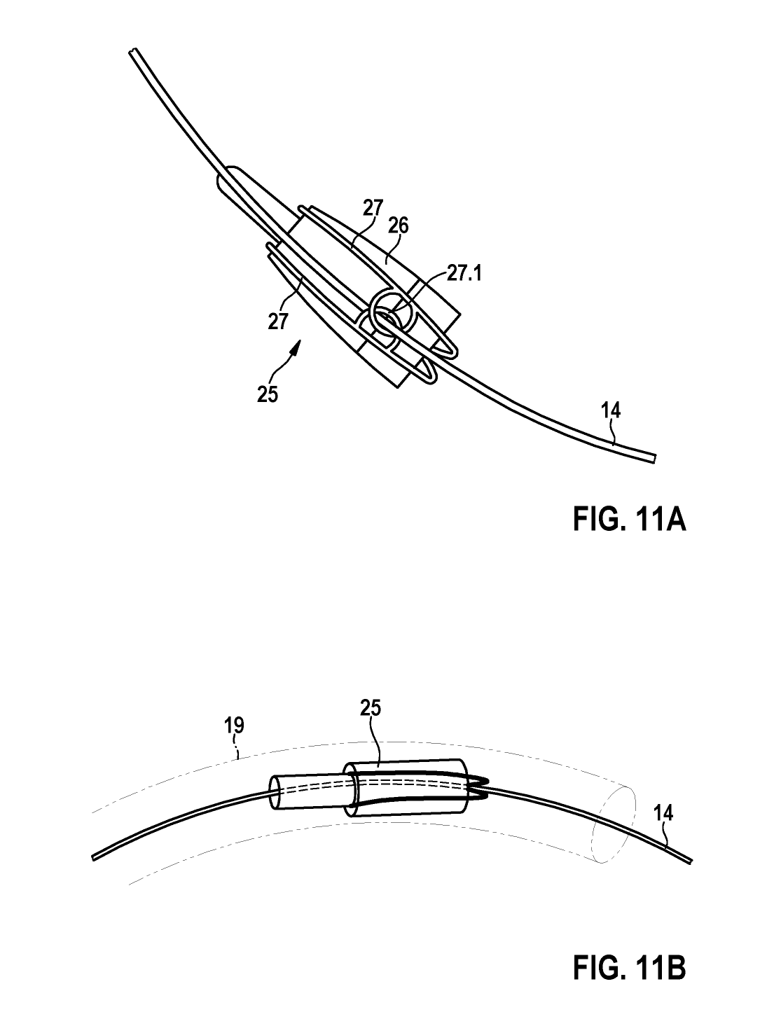

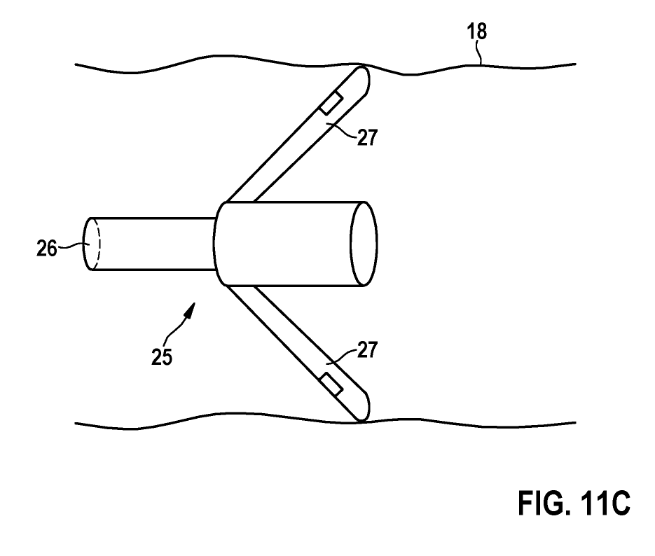

FIGS. 11A-11C show another embodiment of the medical implant 25 and of the system for intravascular implantation of a medical implant 25. What are shown are an implant 25 containing an implant housing 26, a fixing structure 27 with two limbs in the form of arches, and gripper units 27.1, which are arranged on the arches. FIG. 11A shows the embodiment of the implant 25 with the fixing structure 27 in the collapsed state, wherein the gripper units 27.1 have received a guide wire 14, so that the implant 25 is movable along the guide wire. FIG. 11B shows a possible structure for implantation of the embodiment of the implant 25, wherein the implant 25 is arranged on the guide wire 14 within a flexible shaft 19. Possible processes for implantation of the implant 25 are similar to the previously described methods for implantation. FIG. 11C shows the embodiment of the medical implant 25 fixed in a lumen 18 (for example in a blood vessel). Here, the fixing structure 25 is in the expanded form, i.e. the arches are folded out and support the implant housing 26 within the lumen 18.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

The following is a summary list of reference numerals and the corresponding structure used in the above description of the invention:
10 medical implant
11 implant housing
12 fixing structure
12.1, 12.4, 12.5 holder units
12.2 meandering portion
12.3 zigzagging portion
13, 13′ conveying elements
13.1 . . . 13.6 holder members
14 guide wire
15 mechanical spring
16 direction of rotation of the conveying elements
17, 18′ rotation axis of the conveying elements
18 lumen
19 flexible shaft
20 portion protruding from shaft
21 blood vessel
22 portion disposed within the shaft
23.1 slider unit
23.1 slider shaft
23.2, 23.3, 23.4 slider module
23.5, 23.6 bushings
24 gripper unit
25 medical implant
26 implant housing
27 fixing structure
27.1 holder units
28.1, 28.2 angle between limb and blood flow direction
29 blood flow direction

1. A medical implant for intravascular implantation, comprising:
an implant housing; and
at least a first conveying element and a second conveying element, said first conveying element and said second conveying element disposed on said implant housing in one plane, said first conveying element is mounted rotatably about a first rotation axis and said second conveying element is mounted rotatably about a second rotation axis.

2. The medical implant according to claim 1, wherein:
said implant housing has an elongate form with a proximal end and a distal end;
said first conveying element is disposed close to said proximal end; and
said second conveying element is disposed close to said distal end.

3. The medical implant according to claim 1, wherein said first conveying element and the second conveying element are disposed on at least one side outer surface of said implant housing.

4. The medical implant according to claim 1, wherein:
said first rotation axis and said second rotation axis are oriented parallel to one another; and/or
said first rotation axis and said second rotation axis are oriented perpendicular to a longitudinal axis of said implant housing; and/or
said first rotation axis and said second rotation axis are oriented perpendicular to a plane in which said first conveying element and said second conveying element are disposed.

5. The medical implant according to claim 1, wherein said first and the second conveying elements are configured to interact with a guide wire.

6. The medical implant according to claim 5, wherein said first and second conveying elements each have a holder member, and said holder member is configured to receive the guide wire.

7. The medical implant according to claim 6, wherein said holder member has a form of a ring, an eyelet, a loop or a hook.

8. The medical implant according to claim 1, further comprising a fixing structure having at least two holder units, said fixing structure can adopt an expanded state and a collapsed state, and said fixing structure in the expanded state is configured to fix the medical implant at an implantation site.

9. The medical implant according to claim 8, wherein said holder units of said fixing structure are configured to interact with a guide wire, so that said fixing structure is in the collapsed state.

10. The medical implant according to claim 8, wherein said fixing structure contains at least two limbs made of a flexible material, said limbs are closed in the collapsed state, and are folded out from one another in the expanded state, and wherein each of said limbs has a meandering and/or zigzagging portion.

11. The medical implant according to claim 8, further comprising at least one mechanical spring and said fixing structure is coupled to said at least one mechanical spring, said mechanical spring is tensioned when said fixing structure is in the collapsed state, and said mechanical spring is in a rest position when said fixing structure is in the expanded state.

12. The medical implant according to claim 8, wherein said fixing structure is made at least in part of a shape-memory material.

13. The medical implant according to claim 8, wherein said holder unit has a form of a ring, an eyelet, a loop or a hook.

14. The medical implant according to claim 8, wherein said fixing structure is made at least in part of nitinol.

15. A system for intravascular implantation of a medical implant, the system comprising:
a flexible guide wire;
a slider unit having a flexible shaft with a lumen, wherein the medical implant, said flexible guide wire and said slider unit are disposed such that said flexible guide wire interacts with first and second conveying elements of the medical implant, wherein the first and second conveying elements each have a holder member, which holder members receive said flexible guide wire; and
said flexible guide wire is disposed at least in part in said lumen of said flexible shaft of said slider unit, said slider unit is configured to push and/or to pull the medical implant along said flexible guide wire.

16. The system according to claim 15, wherein:
the medical implant further has a fixing structure with at least two holder units, the fixing structure can adopt an expanded state and a collapsed state, the holder units are configured to interact with said flexible guide wire in that said flexible guide wire is received by the holder units, so that the fixing structure is in the collapsed state, and wherein the fixing structure is configured to transition into the expanded state as soon as said flexible guide wire ceases to interact with the holder units.

17. A system for intravascular implantation, comprising:
a medical implant having first and second conveying elements, said first and second conveying elements each having a holder member;
a flexible guide wire;
a slider unit having a flexible shaft with a lumen, wherein said medical implant, said flexible guide wire and said slider unit are disposed such that said flexible guide wire interacts with said first and second conveying elements of said medical implant;
said holder member receives said flexible guide wire; and
said flexible guide wire is disposed at least in part in said lumen of said flexible shaft of said slider unit, said slider unit is configured to push and/or to pull said medical implant along said flexible guide wire.

18. The system according to claim 17, wherein:
said medical implant further has a fixing structure with at least two holder units, said fixing structure can adopt an expanded state and a collapsed state, said holder units are configured to interact with said flexible guide wire in that said flexible guide wire is received by said holder units, so that said fixing structure is in the collapsed state, and wherein said fixing structure is configured to transition into the expanded state as soon as said flexible guide wire ceases to interact with said holder units.
